(19) United States
(12) Patent Application Publication
(10) Pub. No.: US 2011/0053837 A1
(43) Pub. Date: Mar. 3, 2011

(54) Mineral-peptide chelates

(75) Inventors: Shann-Tzong Jiang, Taipei (TW); Li-Jung Yin, Kaohsiung City (TW); Hsueh-Ming Tai, Hsinchu City (TW)

(73) Assignee: Nugen Bioscience (Taiwan) Co., Ltd., Taipei (TW)

(21) Appl. No.: 12/659,241

(22) Filed: Mar. 2, 2010

(30) Foreign Application Priority Data
Sep. 1, 2009 (TW) 098129398

(51) Int. Cl. A61K 38/08; C07K 5/06; C07K 5/08; C07K 5/10; C07K 7/06; A61K 38/05; A61K 38/06; A61K 38/07; C12P 21/06

(52) U.S. Cl. 514/5.4; 530/328; 530/329; 530/330; 530/331; 435/68.1; 514/21.6; 514/21.7; 514/21.8; 514/21.9; 514/21.91

(57) ABSTRACT

The present invention provides a mineral-peptide chelate comprising a peptide consisting of 2˜18 amino acids and a mineral chelated to the peptide, wherein the peptide can be a hydrolysate obtained by hydrolyzing soybean or other protein materials with proteases, or a product obtained by hydrolyzing soybean or other protein material with proteases and fermentation. The mineral-peptide chelate of the present invention may further comprise a carrier which covers the peptide and the mineral which is chelated to the peptide.

### FIELD OF THE INVENTION

The present invention relates to a mineral-peptide chelate, for example, a mineral-peptide chelate comprising a peptide which is a hydrolysate obtained by hydrolyzing soybean or other protein materials with proteases. The absorption and utilization of the mineral-peptide chelate in the gastrointestinal (GI) tract are high.

### BACKGROUND OF THE ART

Complexation and chelation are different. In Complexation, an organic acid is bonded to a mineral via a single bond. In chelation, a divalent metal cation (for example, Mg²⁺, Ca²⁺, Fe²⁺ or Zn²⁺) is partially bonded to the carboxyl anion and amide group of amino acids to form coordinate bondings. The divalent metal cation is “chelated” to the amino acids. In the case, a divalent metal ion is chelated to a protease-hydrolyzed peptide (consisting of several amino acids), a stable cyclic structure is formed by one or more radicals on the peptide with a metal ion via coordination reaction, and the product is a chelated mineral. It was known that minerals can be protected by bonding to absorbable organic molecules, such as amino acids, proteins, polysaccharides and volatile fatty acids. In comparison with complexed minerals or inorganic minerals, chelated minerals are more stable since the bonding strengths in chelated minerals are higher. Chelated minerals therefore can be effectively absorbed by the human or animal body without being first digested and destroyed by the gastric liquid.

The most effective ion transport in the GI tract is well known as active transport, and the absorption of peptides is of this type. Active transport does not require a concentration gradient, but requires energy. Minerals in the form of chelated minerals can be absorbed by the cells.

The National Nutritional Foods Association (NNFA) defined a good chelate of amino acids with a metal element as one having an average molecular weight of less than 800 daltons (Da), which is a product resulting from the chelation between a metal ion and about 3 amino acids.

Most of the commercially available chelated minerals comprise one single species of amino acid. However, an excess uptake of one single species of amino acid inhibits the uptake of other amino acids, thereby leads to an imbalance of uptake of amino acids and also impacts the absorption of minerals chelated to the amino acids.

### SUMMARY OF THE INVENTION

The present invention provides a mineral-peptide chelate comprising a peptide consisting of 2˜18 amino acids and a mineral chelated to the peptide, wherein the peptide can be a hydrolysate obtained by hydrolyzing soybean or other protein materials (such as rice and fish) with proteases, or a product obtained by hydrolyzing soybean or other protein materials (such as rice and fish) with proteases and fermentation. The mineral-peptide chelate of the present invention may further comprise a carrier for covering the peptide and the mineral which is chelated to the peptide.

The present invention also provides a process for the preparation of a mineral-peptide chelate, comprising hydrolyzing a protein material, such as soybean or other protein materials (such us rice and fish) with proteases, fermenting the hydrolyzed protein material, adding a mineral, and spray drying the product to obtain a mineral-peptide chelate.

The soybean as the starting material after being hydrolyzed by proteases are degraded into different peptide fragments. According to prior studies, peptide fragments consisting of 2˜6 amino acids are most easily absorbed by the GI tract and thus enhance the absorption of the minerals chelated to the peptide fragments.

### BRIEF DESCRIPTION OF THE DRAWINGS

FIG. 1 shows the contents of glucoside isoflavones and aglycone isoflavones in each of soybean protein, hydrolyzed soybean protein, and hydrolyzed and fermented soybean protein.

FIG. 2 shows the FTIR spectrum of a Ca-soybean peptide chelate at a degree of chelation of 20% (20% Ca²⁺), wherein y-axis represents absorbance and x-axis represents wavenumbers.

FIG. 3 shows the FTIR spectrum of a Zn-soybean peptide chelate at a degree of chelation of 20% (20% Zn²⁺), wherein y-axis represents absorbance and x-axis represents wavenumbers.

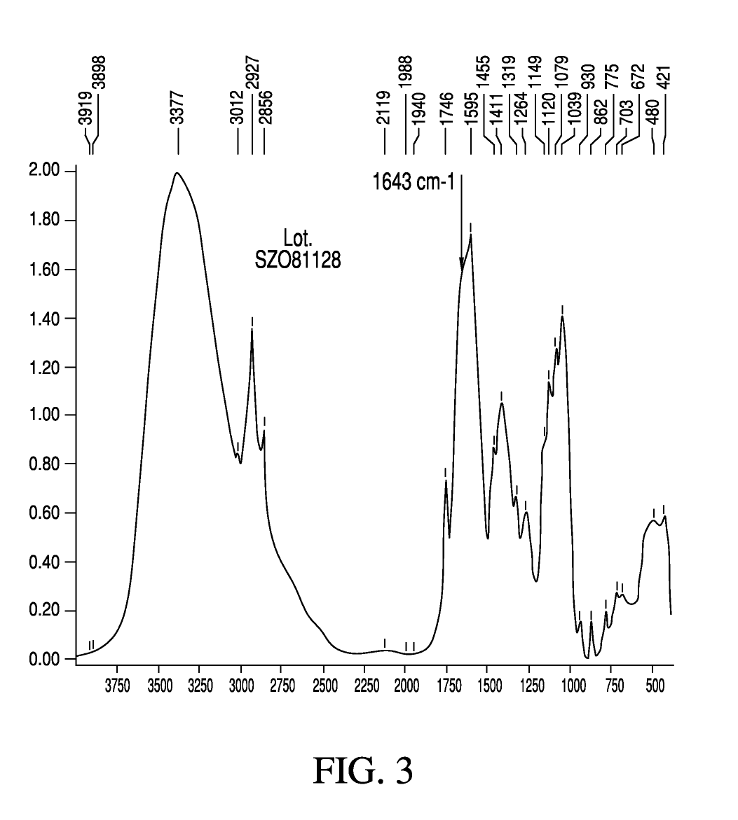

FIG. 4 shows the FTIR spectrum of an Mg-soybean peptide chelate at a degree of chelation of 15% (15% Mg²⁺), wherein y-axis represents absorbance and x-axis represents wavenumbers.

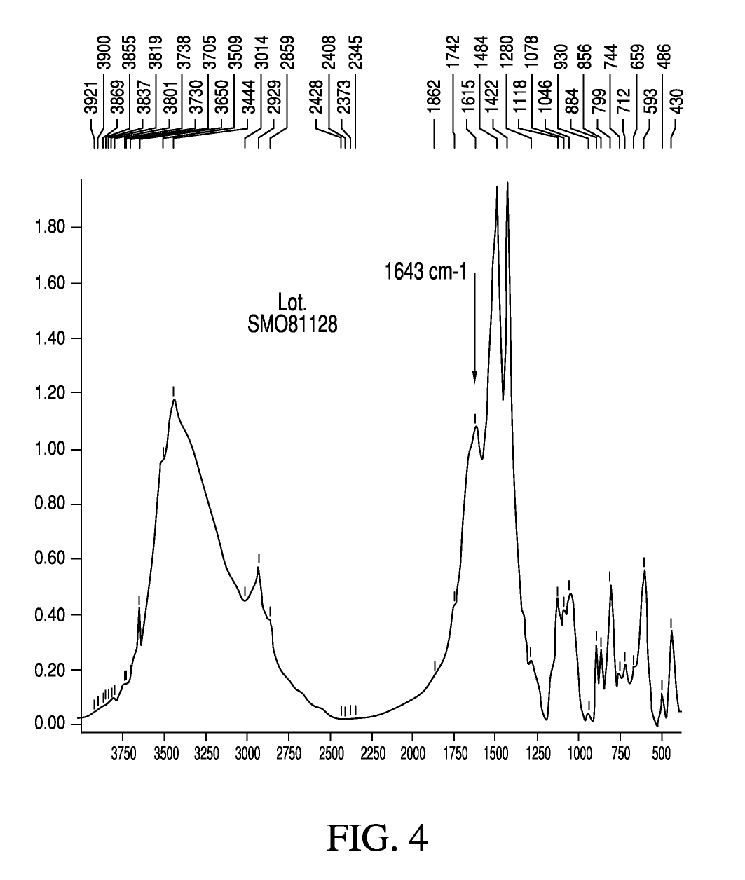

FIG. 5 shows the FTIR spectrum of a Fe-soybean peptide chelate at a degree of chelation of 15% (15% Fe²⁺), wherein y-axis represents absorbance and x-axis represents wavenumbers.

FIG. 6 shows the correlation between the FTIR spectrum and structure of a mineral-peptide chelate at a specific degree of chelation, wherein the peak absorption at 1643 cm⁻¹ is the evidence of ring formation caused by chelation.

FIG. 7 shows the correlation between the FTIR spectra of Ca-soybean peptide chelates and the degrees of Ca chelation of from 0 to 10%, wherein y-axis represents absorbance and x-axis represents wavenumbers.

### DETAILS OF THE INVENTION

In an embodiment of the present invention, natural and non-transgenic soybean as the starting material is hydrolyzed by a selective protease obtained from fermentation of screened Bacillus subtilis. The Bacillus subtilis used for hydrolyzing soybean can be a commercially available product, such as Bacillus subtilis YJ-1. The proteases have hydrolytic activity on the peptide bonds in general proteins so that the proteins can be degraded into small-molecule peptides. The protease is stable at 30˜50° C. (with the best performance at 50° C.) and pH 7˜10 (with the best performance at pH 9).

The hydrolysate obtained by hydrolyzing the soybean with proteases can be further fermented, supplied with minerals and spray dried into mineral-peptide chelates. Commercially available Lactobacilli, such as Lactobacillus acidophilus, can be used in the fermentation step.

The mineral (or metal) source useful for supplying minerals may be a compound of the metal to be added, for example, metal citrates, hydroxides, lactates, oxides, chlorides, carbonates, sulfates, gluconates and the like. The mineral source may be the metal compounds or their aqueous solutions, suspensions or the like. The species of the metals are not limited, depending on the purpose of the metal-peptide chelates, for example depending on the nutrition needed. The metals may include, for example, calcium, zinc, magnesium, iron and the like.

After adding minerals, the resulting semi-product can be covered by a carrier, i.e., be spray dried together with a carrier, to form the final product. The carrier used may be any pharmaceutically acceptable carrier, such as dextrin, lactose, arabinose, polyethylene glycol, sorbitol, hydroxypropyl methylcellulose, microcrystalline cellulose and the like. The final product is a chelate of soybean peptides with minerals, and has features or advantages as explained below:

1. Small-molecule peptides having molecular weights of 500˜2000 Da are formed by hydrolyzing soybean protein with an enzyme, wherein the peptides have molecular weights lower than that of proteins and thus are easily absorbed by the human GI tract and have higher bioavailability.
2. In the product, the profitable amino acid components of soybean are retained, and the contents of the components which inhibit digestive enzymes and the components which cause flatulence are significantly lowered.
3. The chelates of soybean-derived peptides with minerals are rich in soybean isoflavones which are in the easily absorbable aglycone form. In details, general soybean isoflavones include daidzin, genistin, daidzein and genistein, wherein daidzin and genistin are in the glucoside form, and daidzein and genistein are in the aglycone form. The properties of these four substances are listed below:

| | | | |
|---|---|---|---|
| | molecular | | |
| isoflavones | weight | absorption | bioavailability |

glucoside isoflavones (glucoside form)

| | | | |
|---|---|---|---|
| daidzin | high | slow | 20% |
| genistin | high | slow | 20% |

aglycone isoflavones (aglycone form)

| | | | |
|---|---|---|---|
| daidzein | low | fast | 90% |
| genistein | low | fast | 90% |

Soybean proteins which have not been hydrolyzed and fermented contain significantly higher amounts of daidzin and genistin, whilst soybean proteins which have been hydrolyzed and fermented contain significantly higher amounts of daidzein and genistein.
4. Prior studies proved that soybean peptides help stabilize blood pressure.
5. The product is low antigenic and contains no allergenic ingredients, for the reason that the soybean proteins after being hydrolyzed by a protease are degraded into smaller molecules having reduced antigenicity.
6. The chelated peptide molecules carry nutritional substances and thus the absorption of the nutritional substance by the GI tract via active transport can be increased.
7. The mineral substances after being chelated to peptides become much more absorbable by the GI tract than the unchelated mineral substances, and also the product is completely water-soluble. The product which is a chelate of short-chain peptides with minerals is a good mineral source for vegetarians or those having special nutritional requirements.

### EXAMPLES

### Materials and methods

Commercially available strains of Bacillus subtilis YJ-1 were used in the hydrolysis of soybean protein, and commercially available strains of Lactobacillus acidophilus were used in the fermentation after the hydrolysis. DIFCO 0369, a tryptic soy broth (TSB) made by Difco Laboratories of soybean protein was used as the medium for the cultivation of Bacillus subtilis YJ-1; and DIFCO 0881, a de Man, Rogosa, Sharpe medium, made by Difco Laboratories was used for the cultivation of Lactobacillus acidophilus.

(1) Cultivation of Bacillus subtilis YJ-1
(a) Bacillus subtilis YJ-1 at −80° C. was added into a fresh 10 ml TSB and cultivated at 37° C. for 24 hours to activate the Bacillus subtilis YJ-1.
(b) 1% of the bacteria liquid obtained in the above step (a) was added into a fresh 10 ml TSB and cultivated at 37° C. for 24 hours.
(c) 0.3 ml of the bacteria liquid obtained in the above step (b) was inoculated into a fresh 30 ml TSB and cultivated at 37° C. with agitation for 12 hours. The resulting culture was inoculated into a fermentation tank for large-scale cultivation.
(d) The liquid culture thus formed was spray-dried to remove water and obtain protease powder.
(2) Cultivation of Lactobacillus acidophilus
(a) Lactobacillus acidophilus at −80° C. was added into a fresh 10 ml MRS and cultivated at 37° C. for 24 hours to activate the Lactobacillus acidophilus.
(b) 1% of the bacteria liquid obtained in the above step (a) was added into a fresh 10 ml MRS and cultivated at 37° C. for 24 hours.
(c) 5 ml of the bacteria liquid obtained in the above step (b) was inoculated into a fresh 500 ml MRS and cultivated at 37° C. for 24 hours.

In each of the above methods (1) concerning cultivation of Bacillus subtilis YJ-1 and (2) concerning cultivation of Lactobacillus acidophilus, before the inoculation of Bacillus subtilis YJ-1 or Lactobacillus acidophilus, all the mediums must be first sterilized at 121° C. for 15 minutes to insure the desired strains are solely cultivated in a sterile condition.

(3) Steps for preparing mineral-peptide chelates
1. Soybeans were soaked in tap water for 16 hours, and the soaked soybeans and water were ground by the action of a grinder, then were boiled at 100° C. for 30˜45 minutes.
2. The boiled soybean liquid was cooled to 50° C., the proteases obtained by the above method (1) were added therein and stirred homogeneously to carry out the hydrolysis at 50° C. for 6˜10 hours, with continuous stirring during the hydrolysis.
3. Lactobacillus acidophilus was added to the soybean hydrolysate obtained in the above method (2) in an amount according to the volume of the hydrolysate, and was stirred homogeneously. The culture was allowed to stand for 12-48 hours at 37° C. and the pH values before and after fermentation were monitored.
4. The pH was adjusted and mineral sources were added. If calcium is to be added, the mineral source can be calcium citrate, calcium hydroxide or calcium lactate; if magnesium is to be added, the mineral source can be magnesium oxide, magnesium chloride or magnesium carbonate; if zinc is to be added, the mineral source can be zinc sulfate, zinc oxide or zinc gluconate; if iron is to be added, the mineral source can be ferrous lactate or ferrous gluconate.
5. The semi-product thus obtained was adjusted to having a neutral pH, and was spray-dried with dextrin, lactose or arabinose which acts as a carrier.

The molecular weight of the soybean peptide hydrolysate thus obtained was determined by HPLC. The results were that the number of small molecular segments was significantly increased, and the peptides after hydrolysis have molecular weights of 500˜2500 Da. Compared with soybean protein, these small molecular segments peptides are easy to absorb and have higher bioavailability.

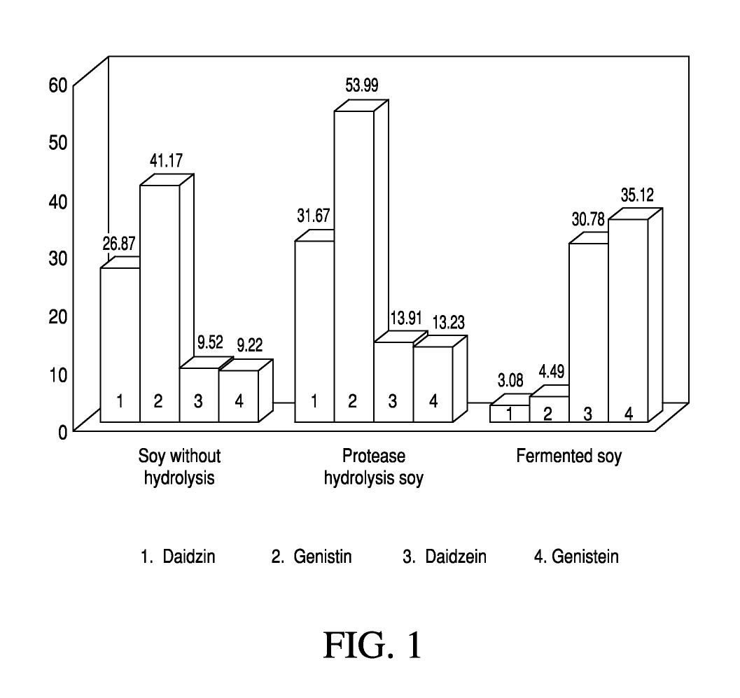

During the production process, the contents of glucoside isoflavones (including daidzin and genistin) and aglycone isoflavones (including daidzein and genistein) were determined, and the results are shown in FIG. 1. It is apparent from FIG. 1 that the hydrolyzed and fermented soybean peptides contain significantly more daidzein and genistein.

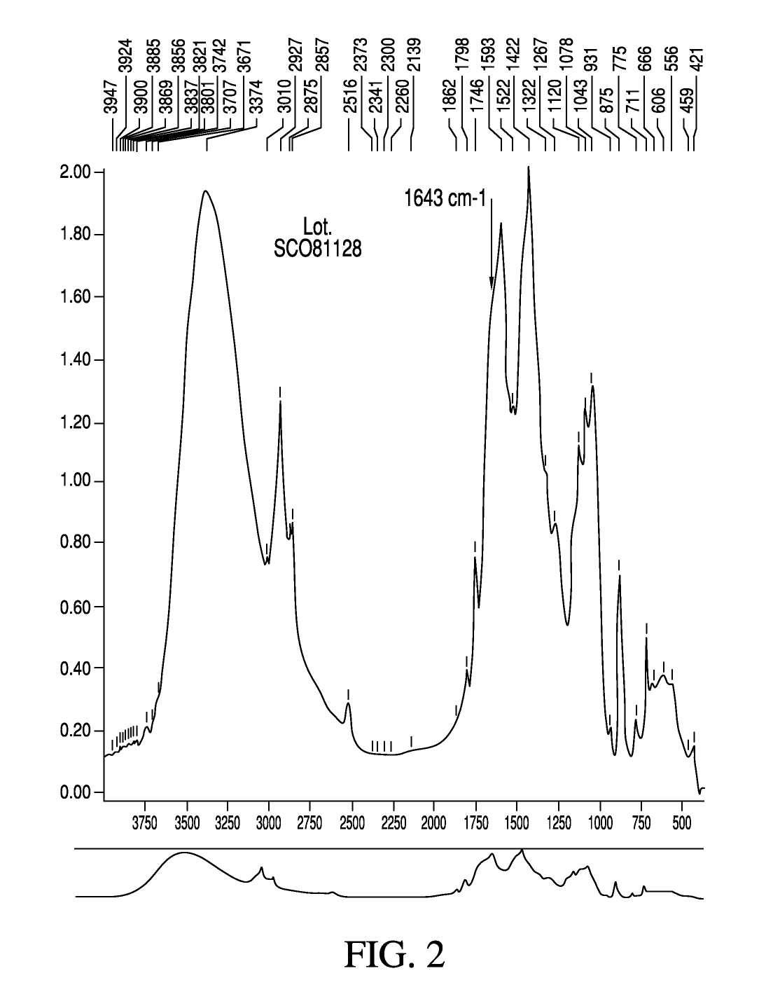

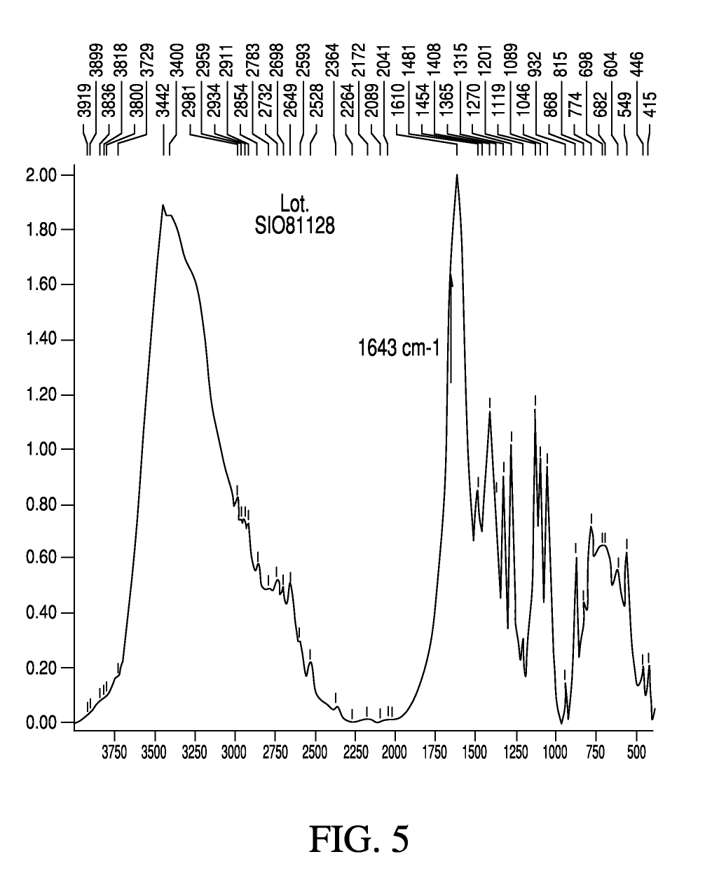

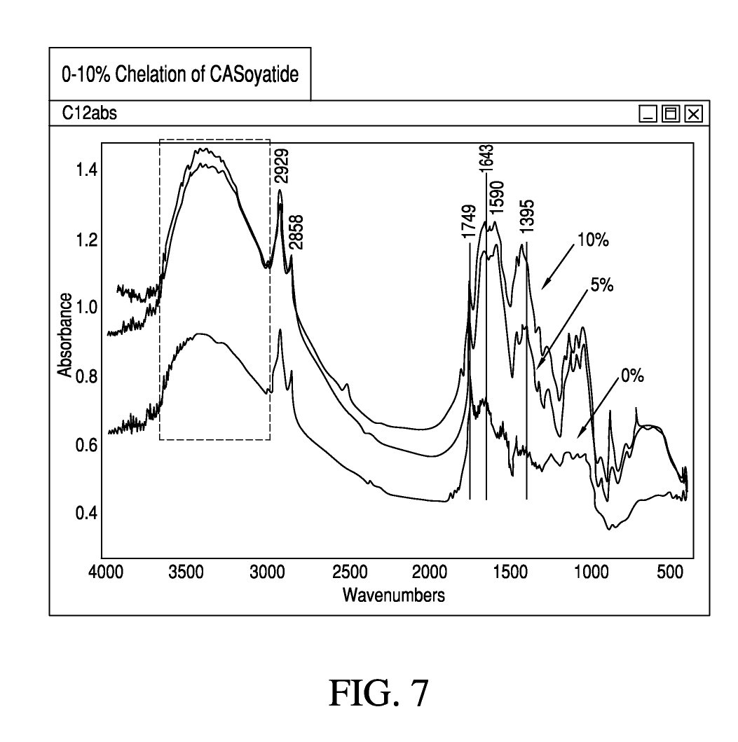

Degrees of metal chelation were determined by using a Fourier Transform Infrared spectrometer (FTIR spectrometer) at 1643 cm⁻¹ and are shown in FIGS. 2˜FIG. 5. Further as shown in FIG. 7, when degree of Ca chelation is increased from 0 to 10%, the absorbance at wavenumber (or frequency) 1643 cm⁻¹ also is increased, meaning that more minerals are chelated to peptides.

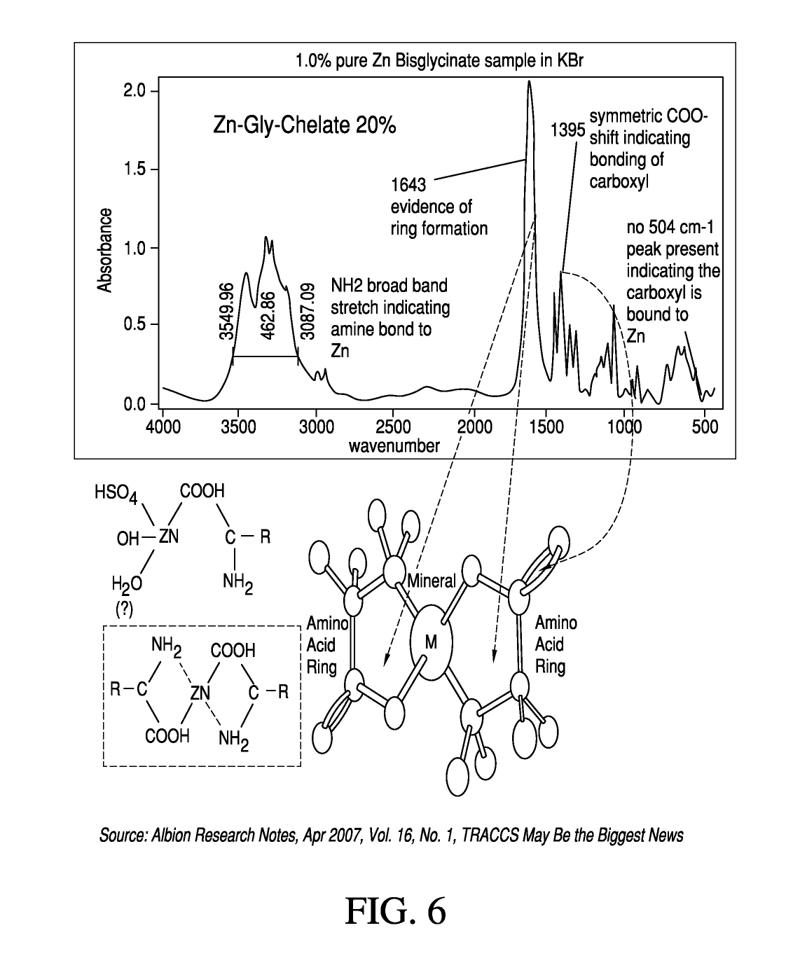

Degrees of metal chelation were particularly determined at wavenumber 1643 cm⁻¹ by using a FTIR spectrometer for the reason that ring structures are formed if chelation occurs, as shown in FIG. 6, wherein a peak absorption at 1643 cm⁻¹ is the evidence for ring formation. The COO⁻ groups bind to the minerals to become a part of the ring, causing bending and stretching of the C═O moieties of the COO⁻ groups and an increase of absorbance at the wavenumber of about 1395 cm⁻¹. Also, an increase of absorbance is caused at the wavenumber where a change of amide groups occurs.

Table 1 shows the differences of specific wavenumbers in the FTIR spectra between an amino acid and a metal-oligopeptide chelate. FIG. 6 can support the data in Table 1.

TABLE 1
the differences of specific wavenumbers in the FTIR spectra between an
amino acid and a metal-oligopeptide chelate.

| Glycine | | Zinc bisglycinate crystals | |
|---|---|---|---|
| Wavenumber | | Wavenumber | |
| (cm⁻¹) | Assignment | (cm⁻¹) | Assignment |
| 3050-2675 | NH³* broad bands | 3550-3050 | NH² broad bands |
| 1410 | Symmetric COO⁻ | 1395 | Symmetric COO⁻ |
| | stretch | | stretch |
| 504 | COO⁻ rock | [symbol] | Evidence of ring |
| | | | formation |

Soybean peptides and Ca-soybean peptide chelates are analyzed by HPLC, and the results are that most of them have molecular weights of lower than 800 Da. Other similar commercially available products all have molecular weights of much higher than 800 Da.

What is claimed is:

1. A mineral-peptide chelate comprising a peptide consisting of 2˜18 amino acids and a mineral chelated to the peptide.

2. The mineral-peptide chelate of claim 1, wherein the peptide can be a hydrolysate obtained by hydrolyzing soybean or other protein materials (such as rice and fish) with proteases.

3. The mineral-peptide chelate of claim 1, wherein the peptide can be a product obtained by hydrolyzing soybean or other protein material (such as rice and fish) with proteases and fermentation.

4. The mineral-peptide chelate of claim 1, wherein the mineral is calcium, zinc, magnesium or iron.

5. The mineral-peptide chelate of claim 1, which further comprises a carrier which covers the peptide and the mineral chelated to the peptide.

6. The mineral-peptide chelate of claim 1, wherein the carrier is a pharmaceutically acceptable carrier.

7. The mineral-peptide chelate of claim 1, wherein the carrier is dextrin, lactose, arabinose, polyethylene glycol, sorbitol, hydroxypropyl methylcellulose or microcrystalline cellulose.

8. A process for the preparation of the mineral-peptide chelate defined in claim 1, comprising hydrolyzing soybean or other protein materials (such as rice and fish) with proteases, fermenting the hydrolyzed protein material, adding a mineral, and spray drying the product to obtain a mineral-peptide chelate.

9. The process of claim 8, wherein soybean or other protein materials (such as rice and fish) is hydrolyzed by using proteases obtained from fermentation of Bacillus subtilis.

10. The process of claim 8, wherein the hydrolysate obtained by hydrolyzing the soybean with proteases is fermented by using Lactobacillus acidophilus.

11. The process of claim 8, wherein in the step of adding a mineral a compound of the mineral is used.

12. The process of claim 8, wherein after adding a mineral the resulting semi-product is spray dried together with a carrier.

13. The process of claim 8, wherein the carrier is a pharmaceutically acceptable carrier.

14. The process of claim 13, wherein the carrier is dextrin, lactose, arabinose, polyethylene glycol, sorbitol, hydroxypropyl methylcellulose or microcrystalline cellulose.
